(19) United States
(12) Patent Application Publication
(10) Pub. No.: US 2010/0016707 A1
(43) Pub. Date: Jan. 21, 2010

(54) IMAGING SYSTEM

(75) Inventors: Avi Amara, Katzir-Doar-Na Menashi (IL); Arie Amara, Misgav (IL)

Correspondence Address: MARTIN D. MOYNIHAN d/b/a PRTSI, INC., P.O. BOX 16446, ARLINGTON, VA, 22215, US

(73) Assignee: KETER MEDICAL LTD, KIRYATSHMONA, IL (US)

(21) Appl. No.: 12/310,043

(22) PCT Filed: Jul. 30, 2007
(86) PCT No.: PCT/IL2007/000951

§ 371 Date: Sep. 16, 2009

Related U.S. Application Data
(60) Provisional application No. 60/821,749, filed on Aug. 8, 2006.

(51) Int. Cl. A61B 5/055; A61B 8/00; A61B 5/05

(52) U.S. Cl. 600/411; 600/453; 600/407

(57) ABSTRACT

A medical diagnostic system, the system comprising:

a) a non-ultrasound imaging module configured to generate a volumetric vascular map;

b) a controller configured to translate a 3D position co-ordinate from the map into an aiming instruction;

c) a Doppler ultrasound unit adapted to aim a transducer responsive to the aiming instruction; and

d) a registration module configured to register a position of the transducer with respect to the volumetric map.

### FIELD OF THE INVENTION

The present invention relates to systems and methods for diagnosis of blood flow abnormalities in blood vessels.

### BACKGROUND OF THE INVENTION

A. V. Alexandrov (2004) Stroke 35:2722, the disclosure of which is incorporated herein by reference, describes using diagnostic ultrasound with tissue plasminogen activator (tPA) to effect thrombolytic treatment.

The Doppler Effect, described below, is often used to assess the speed of moving objects or fluids in ultrasonic imaging. There is a relationship between the frequency of a transmitted wave and the frequency of the wave after being reflected from a moving object. A transmitted wave of a given frequency which impinges upon a moving object is reflected with a frequency shift proportional to the velocity of that object in a direction toward or away from the detector. This signal is reflected and backscattered from moving objects with a positive or negative frequency shift, depending of the direction of the backscatter relative to the flow direction. The frequency shift is called the Doppler Effect or Doppler shift and the reflected or scattered signal is usually called the Doppler signal.

Transcranial Doppler Ultrasonography (TCDUS) is a noninvasive technology that uses the Doppler Effect to measure the velocity and direction of blood flow in the vessels. TCDUS typically uses a handheld-pulsed low frequency Doppler transducer that enables recording of blood flow velocities from intracranial arteries through selected cranial foramina and thin regions of the skull. Analysis of the Doppler spectra allows display and calculation of peak systolic, peak diastolic, and mean velocities and pulsatility indices. Mapping of the sampled velocities as a color display of spectra in lateral, coronal and horizontal views locates the major brain arteries in three dimensions. TCDUS obtains information about the physiology of flow through the major basal intracranial arteries by measuring velocities and pulsatilities in segments of these arteries, while PET and SPECT scanning, Xenon enhanced CT scanning, and MRI spectroscopy yield images or quantitative data about metabolism and perfusion of brain regions but do not give direct data about flow in major supplying arteries. Cerebral angiography provides an image of the anatomical configuration of the lesions of the intracranial and extracranial arteries and their proximal, deep and superficial branches.

US published patent application 2004/0138563, describes combining diagnostic ultrasound with therapeutic ultrasound for thrombolysis. Aiming of ultrasonic transducers as disclosed in this application is manual and is based on Doppler data indicative of cerebral blood flow velocity from the transducer. It is not clear how the transducer is accurately aimed at a target throughout the therapy. This application teaches use of a wide therapeutic beam to compensate for low accuracy in aiming. The disclosure of this application is incorporated herein by reference.

US published patent applications 2003/0054027 and 2003/0012735 as well as U.S. Pat. No. 6,231,834 describe improved injectable vesicles containing contrast material for use in non-ultrasound imaging methods including CT angiography. According to disclosures of these references, ultrasound energy causes vesicles of the contrast material to lyse at a target location and contributes to a therapeutic effect. These references are concerned primarily with formulation of the contrast materials and not with means of diagnosing targets. The disclosures of these applications are incorporated herein by reference.

It is also known to use non ultrasound imaging (e.g. CT or MRI) to generate a map for focusing of high energy ultrasound for medical treatment.

WO 2006/001693 describes a system which employs an imaging means (e.g. MRI) to aim ultrasound transducers movably mounted in a frame by means of a gyroscope like construction. However, this application is concerned primarily with targeting ultrasound energy to proliferative tissue, e.g., in the breast. The disclosure of this application is incorporated herein by reference.

U.S. Pat. No. 5,485,839 to Aida describes methods and apparatus for ultrasonic treatment using CT MRI or other topographic means. Aida describes acquiring a CT image and focusing ultrasound energy on targets identified in the CT image. The disclosure of this patent is incorporated herein by reference.

U.S. Pat. No. 6,461,586 describes use of magnetic resonance angiography in identification of a stenotic occlusion in a blood vessel. This patent also discloses use of magnetic resonance angiography to assess thrombolysis induced by a therapeutic ultrasound beam. The disclosure of this patent is incorporated herein by reference.

U.S. Pat. Nos. 6,773,408; 6,618,620; 6,516,211; 6,374,132; 6,128,522 and 5,897,495 describe use of MRI imaging data to aim high energy focused ultrasound. These patents disclose “automatic treatment” after an operator marks targets on the MRI output map. The disclosures of these patents are incorporated herein by reference.

Computerized tomography angiography (CTA) provides volumetric vascular maps with flow data. Principles of CTA are discussed in, for example, in Kretschman's “Cranial Neuroimaging and Clinical Neuroanatomy: Magnetic Resonance Imaging and Computed Tomography”; second edition (1992) ISBN#:1588901459, the disclosure of which is fully incorporated herein by reference.

Functional magnetic resonance induction (MRA) also provides vascular maps with flow data. Principles of MRA (Magnetic Resonance Angiography) are also discussed in the Kretschman reference.

### SUMMARY OF THE INVENTION

A broad aspect of the invention relates to registration of flow information from Transcranial Doppler Ultrasonography (TCDUS) onto a volumetric cerebral vasculature map.

An aspect of some embodiments of the invention relates to use of a non-ultrasound imaging method to produce a high resolution volumetric map of cerebral vasculature and employing Transcranial Doppler Ultrasonography (TCDUS) to provide flow information, optionally hemodynamic flow information, which is registered to the map. In an exemplary embodiment of the invention, a controller translates map coordinates into aiming instructions for one or more TCDUS units. In an exemplary embodiment of the invention, the aiming instructions can be provided as triplets comprising θX (X angle); θY (Y angle) and displacement.

In an exemplary embodiment of the invention, the flow information includes one or both of areas with increased velocity (e.g., due to narrowing) and areas of no velocity (e.g., due to blockage). Optionally, the flow information includes an indication of blood pooling outside of vessels (e.g., where clot dissolution might reopen a hemorrhage). In an exemplary embodiment of the invention, this information is used to determine, optionally automatically, for example depending on one or more thresholds, a desire to treat or not treat a region and/or an expected effect of treatment (e.g., expected time to increase or reduce velocity and expected or desired amount of increase/reduction). Optionally, a table is provided including baseline levels for known anatomical feature sin the brain and/or for particular blood vessel diameters.

Optionally, the non-ultrasound imaging method is computerized tomography (CT) or magnetic resonance induction (MRI).

Optionally, the flow information pertains to a significant portion of, optionally all of, the mapped vasculature.

Optionally, the flow information pertains primarily to one or more selected targets in the map.

According to previously available alternatives, flow information was provided by manual TCDUS imaging of the entire cranium. While this gave good flow data, such imaging can take in excess of one minute. According to some embodiments of the present invention, TCDUS imaging is performed only on suspected targets which are identified by some other method. This gives the relevant diagnostic information in a much shorter time, optionally substantially in real time. Alternatively or additionally, use of a TCDUS unit controlled by an automated controller increases speed and/or accuracy of a return to an identified target.

In an exemplary embodiment of the invention, the non-ultrasound imaging method provides a map which indicates the amount of blood present in the vasculature. The map indicates areas of abnormal flow as abnormal changes in vessel anatomic dimensions. Optionally, the anatomic map is provided by CT angiography (CTA) or MRI angiography (MRA). Optionally, the amount of blood is represented as an amount of contrast material.

In an exemplary embodiment of the invention, analysis of a CTA or MRA map provides a preliminary identification of cerebral blood vessel targets with abnormal flow based upon their anatomic appearance.

In an exemplary embodiment of the invention, the preliminary identification of cerebral blood vessel targets is used to provide aiming instructions through a controller to at least one TCDUS unit so that ultrasound energy is applied to the identified targets.

According to one aspect of some embodiments of the invention, a vasculature map including contrast based flow data, optionally a CTA or MRA map, is registered to the position of a TCDUS transducer, optionally two or more transducers. The TCDUS transducer(s) provides flow hemodynamic information pertaining to blood vessels in the vasculature map and the hemodynamic flow information is employed to identify one or more targets. Optionally, a controller provides aiming instructions to at least one TCDUS unit so that ultrasound energy is applied to the identified targets.

Optionally the identification is performed by analytic circuitry.

Optionally the identification is performed by a human operator, for example by using a computer cursor to indicate areas of the map on a display screen.

In an exemplary embodiment of the invention, each target is translated into a set of aiming instructions for diagnostic TCDUS transducers. Optionally, the transducers are head mounted transducers. Head mounting may be accomplished, for example, by use of a crown, a headband or headset. Optionally, the mounting is selected to overlay thin areas of the skull and/or to ensure coverage of a region of interest in the brain.

Optionally, Doppler ultrasound is concurrently aimed at a single target from two or more directions.

Optionally, TCDUS potentiates thrombolytic activity of a systemically delivered medication. In an exemplary embodiment of the invention, the medication is tPA.

An aspect of some embodiments of the invention relates to use of TCDUS to produce a volumetric map of cerebral vasculature with flow information, identifying targets in the map and providing hemodynamic flow information with respect to the targets based on additional TCDUS analysis. In an exemplary embodiment of the invention, a controller translates map coordinates into aiming instructions for one or more TCDUS units.

Optionally, the flow information pertains to a significant portion of, optionally all of, the mapped vasculature.

There is thus provided in accordance with an exemplary embodiment of the invention, a medical diagnostic system, the system comprising:

a) a non-ultrasound imaging module configured to generate a volumetric vascular map;

b) a controller configured to translate a 3D position co-ordinate from the map into an aiming instruction;

c) a Doppler ultrasound unit adapted to aim a transducer responsive to the aiming instruction; and

d) a registration module configured to register a position of the transducer with respect to the volumetric map.

In an exemplary embodiment of the invention, the system comprises:

e) a target identification module.

Optionally, the target identification module operates automatically to identify targets in the volumetric map.

In an exemplary embodiment of the invention, the target identification module responds to a user indication of targets in the volumetric map.

In an exemplary embodiment of the invention, the controller formulates the aiming instruction responsive to a target identified by the target identification module.

In an exemplary embodiment of the invention, the imaging module employs computerized tomography (CT).

In an exemplary embodiment of the invention, the imaging module employs magnetic resonance induction (MRI).

In an exemplary embodiment of the invention, the imaging module employs CT angiography (CTA).

In an exemplary embodiment of the invention, the imaging module employs MRI angiography (MRA).

In an exemplary embodiment of the invention, the vasculature includes cerebral vasculature.

In an exemplary embodiment of the invention, the Doppler ultrasound unit is a transcranial Doppler ultrasound unit (TCDUS).

In an exemplary embodiment of the invention, the controller is adapted to scan said Doppler according to said map.

In an exemplary embodiment of the invention, the system comprises a medication providing system whose activity is coordinated with said Doppler ultrasound unit.

There is also provided in accordance with an exemplary embodiment of the invention, a medical diagnostic method, the method comprising:

a) generating a volumetric map of a portion of a vasculature system using a non-ultrasound imaging modality;

b) registering a position of a Doppler ultrasound transducer with respect to the volumetric map;

c) identifying a target on the map by a 3D position co-ordinate;

d) translating the 3D position co-ordinate into an aiming instruction;

e) aiming a Doppler ultrasound transducer at the target responsive to the aiming instruction; and

f) acquiring hemodynamic data pertaining to the target.

Optionally, the identifying is performed by analytic circuitry.

Optionally, the identifying is performed by a user.

In an exemplary embodiment of the invention, the volumetric map is generated from computerized tomography (CT) data.

In an exemplary embodiment of the invention, the volumetric map is generated from magnetic resonance induction (MRI) data.

In an exemplary embodiment of the invention, the volumetric map includes blood flow data.

In an exemplary embodiment of the invention, the blood flow data is provided by CT angiography (CTA).

In an exemplary embodiment of the invention, the blood flow data is provided by MRI angiography (MRA).

In an exemplary embodiment of the invention, the volumetric map depicts cerebral vasculature.

In an exemplary embodiment of the invention, the Doppler ultrasound transducer is a transcranial Doppler ultrasound unit (TCDUS).

There is also provided in accordance with an exemplary embodiment of the invention, a therapeutic method, the method comprising:

a) generating a volumetric map of a portion of a vasculature system of a subject using a non-ultrasound imaging modality, the map including preliminary blood flow data;

b) registering a position of a Doppler ultrasound transducer with respect to the volumetric map;

c) identifying a target on the map by a 3D position co-ordinate responsive to the preliminary blood flow data;

d) translating the 3D position co-ordinate into an aiming instruction;

e) aiming a Doppler ultrasound transducer responsive to the aiming instruction; and

f) acquiring hemodynamic data pertaining to the target concurrent with systemic administration of a thrombolytic drug.

There is also provided in accordance with an exemplary embodiment of the invention, an apparatus for reducing signal interference in an ultrasound examination, the apparatus comprising;

a) a cushion adapted to conform to the skull temporal window surface and to an ultrasonic transducer; and

b) a quantity of a first ultrasound coupling media contained within the cushion at a pressure selected to allow the conforming;

wherein the conforming to the skull temporal window surface and the conforming to the ultrasound transducer preclude the formation of air pockets capable of significantly interfering with ultrasonic transmission.

In an exemplary embodiment of the invention, the first ultrasound coupling media includes an oil.

In an exemplary embodiment of the invention, the first ultrasound coupling media includes a gel.

In an exemplary embodiment of the invention, the first ultrasound coupling media includes water.

In an exemplary embodiment of the invention, the apparatus comprises:

a second ultrasound coupling media applied to the first surface.

In an exemplary embodiment of the invention, the apparatus comprises:

a second ultrasound coupling media applied to the second surface.

In an exemplary embodiment of the invention, the second ultrasound coupling media includes a gel.

In an exemplary embodiment of the invention, the apparatus comprises an ultrasound transducer ultrasonically coupled to said cushion and a motor adapted to physically move said ultrasonic transducer.

There is also provided in accordance with an exemplary embodiment of the invention, a medical diagnostic system, the system comprising:

a) a transcranial Doppler ultrasound unit including at least one transducer, the unit attached to a cranium and adapted to scan a brain volume and generate a 3D volumetric vascular map of at least part of the cranium;

b) a controller configured to translate a 3D position co-ordinate from the map into an aiming instruction; and

c) an aiming mechanism adapted to aim a transcranial Doppler transducer responsive to the aiming instruction.

### BRIEF DESCRIPTION OF THE DRAWINGS

Exemplary non-limiting embodiments of the invention described in the following description, read with reference to the figures attached hereto. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a simplified flow diagram of a method according to exemplary embodiments of the invention;

FIG. 2 is a schematic representation of a system according to exemplary embodiments of the invention;

FIG. 3A is a perspective view of a headset including two transcranial Doppler ultrasound units according to an exemplary embodiment of the invention;

FIG. 3B is a cross section of a transcranial Doppler ultrasound unit according to an exemplary embodiment of the invention;

FIG. 4 is a transverse longitudinal section of the brain with a vascular map and ultrasound unit according to an exemplary embodiment of the invention superimposed thereon;

FIG. 5 is a vascular map with an identified target indicated according to an exemplary embodiment of the invention;

FIG. 6 illustrates a Doppler ultrasound output from a target as depicted in FIG. 4; and

FIGS. 7A; 7B and 7C illustrate a series of Doppler ultrasound outputs from a target as depicted in FIG. 4 at times 12 minutes; 12.5 minutes and 6 hours from onset of drug therapy respectively.

### DETAILED DESCRIPTION OF EXEMPLARY EMBODIMENTS

### Exemplary Diagnostic Method

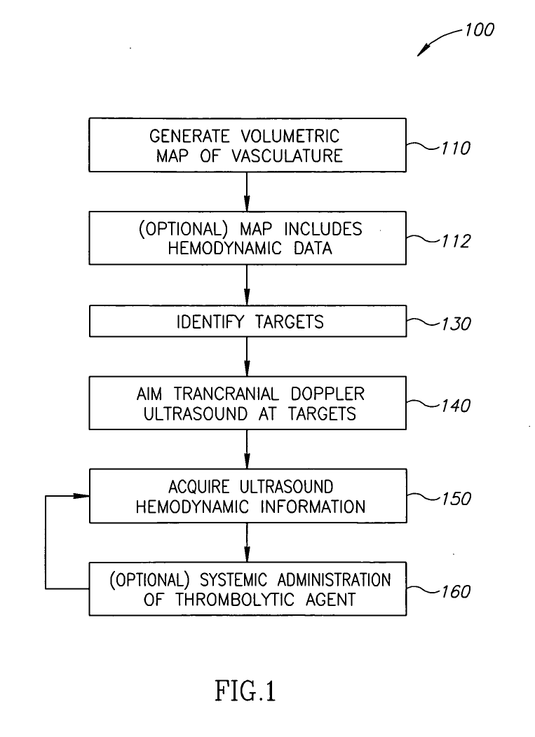

FIG. 1 is a simplified flow diagram illustrating a series of actions associated with a diagnostic imaging method 100 according to exemplary embodiments of the invention.

At 110 a volumetric map of the vasculature is generated, by a non-ultrasound means. The non-ultrasound means may be, for example computerized tomography (CT); magnetic resonance induction (MRI), CT angiography (CTA) or MRI angiography (MRA).

The map generated at 110 includes blood vessel dimension data. Optionally, blood vessel dimensions may be ascertained by an amount of contrast material in each vessel. In some embodiments of the invention, areas of abnormal narrowing in a blood vessel are interpreted as areas of reduced flow. Optionally, the data includes both actual dimension and expected dimension, for example, based on nearby segments of the blood vessel or based on identification of the blood vessel and comparing to an anatomical atlas.

In an exemplary embodiment of the invention, TCDUS volumetric scanning data is acquired and registered with respect to the map produced at 110 to produce 112 a map with blood flow (velocity) data associated with each blood vessel. Optionally, superimposition of blood flow data on anatomic data provides a direct correlation between anatomic abnormalities and flow abnormalities.

In an exemplary embodiment of the invention, TCDUS is conducted only along blood vessels identified in the map by non-ultrasonic means. In an exemplary embodiment of the invention, this reduces the amount of tissue scanned by TCDUS by as much as 30, 40, 50, 60, 70 or 80%. This can result in a time savings of as much as 30, 40, 50, 60, 70 or 80%.

Once the map has been produced, identification 130 of targets is conducted. Optionally, targets are areas characterized by a reduction in, or absence of, blood flow as analyzed by TCDUS. Optionally, targets are areas characterized by an atypical narrowing of vessels. In an exemplary embodiment of the invention, targets are characterized as locations where a reduction in blood flow and atypical narrowing of vessels coincide. It is noted that a reduction in blood diameter, up to a point, is often indicated by an increased velocity, except that a total or near-total occlusion usually has a near zero velocity.

In an exemplary embodiment of the invention, the map is analyzed automatically by analytic circuitry to identify targets. Alternatively or additionally, the map is presented on a display screen and targets are selected manually. Whether selection is automatic or manual, identified targets can be defined in terms of position co-ordinates.

Once targets have been identified 130, aiming 140 of TCDUS at the targets is performed.

Acquisition 150 of blood flow information for each target is performed using TCDUS. In an exemplary embodiment of the invention, this flow information is quantitative. Optionally, the flow information is presented as a numerical flow rate and/or a graph of flow vs. time.

Optionally, acquisition 150 of hemodynamic flow information by TCDUS is safer than acquisition of blood flow information by non-ultrasound means.

For example, use of TCDUS may reduce exposure to ionizing radiation employed in CT or to a RF and a magnetic field employed in MRI. Alternatively or additionally, TCDUS does not rely upon contrast agents, many of which are potentially toxic and/or elicit allergic reactions.

Use of TCDUS optionally permits analysis of hemodynamic flow information over a long period of time (e.g., a period of several hours). Use of non-ultrasound means over a similar period of time could require repeated administration of contrast reagents.

In an exemplary embodiment of the invention, hemodynamic flow information acquired 150 by TCDUS is qualitatively different than flow information acquired by non-ultrasound means. TCDUS provides flow information for a specific point as a directional velocity (e.g. in cm/s). Adding this flow data to a vascular map produced by CTA or MRA by increases the value of information by correlating a physiologic parameter with blood vessel diameter. It is noted that for some vessel orientations, the measured velocity may be ambiguous. Optionally, this is overcome by using multiple transmitters, or by combining an indication from the non-TCDUS modality with the signal from the TCDUS modality to obtain a probability of a location requiring treatment.

Alternatively or additionally, combination of TCDUS hemodynamic flow data with a vascular map produced by non-ultrasound means can increase resolution. For example, CTA image resolution may be as exact as ±1 mm, while resolution of flow information provided by TCDUS may be as inexact as ±5 mm or less exact. Registration of TCDUS data onto a vascular map produced by CTA correlates flow data to a location in a specific vessel.

In an exemplary embodiment of the invention, analysis of maximum flow data provided by TCDUS pinpoints a stenosis, even if no anatomic abnormality is discernible on the map.

In an exemplary embodiment of the invention, aiming 140 permits acquisition 150 without scanning and contributes to speed and/or accuracy.

Optionally, TCDUS potentiates thrombolytic activity of a systemically administered 160 medication. In those embodiments of the invention which employ a systemically administered medication, aiming 140 at targets and acquiring 150 hemodynamic information with respect to the targets is repeated while the medication is in the blood stream. Optionally, the timing of imaging is dependent on the timing of delivery of a medication, for example, to ensure that imaging and/or ultrasonic irradiation are performed at a time when progress is expected and/or an interaction between the radiation and a medication is expected.

In an exemplary embodiment of the invention, the medication is a thrombolytic medication (e.g. tPA) delivered to promote recanalization. Use of thrombolytic agents in general and tPA in particular is well known in the art. In an exemplary embodiment of the invention, TCDUS provides diagnostic information concerning a degree of recanalization achieved by the medication and/or TCDUS and/or a combination thereof. Interaction between tissue plasminogen activator is well known and has been described for example by A. V. Alexandrov in “Ultrasound Identification and Lysis of Clots” ((2004) Stroke 35:2722), the disclosure of which is fully incorporated herein by reference.

Exemplary System

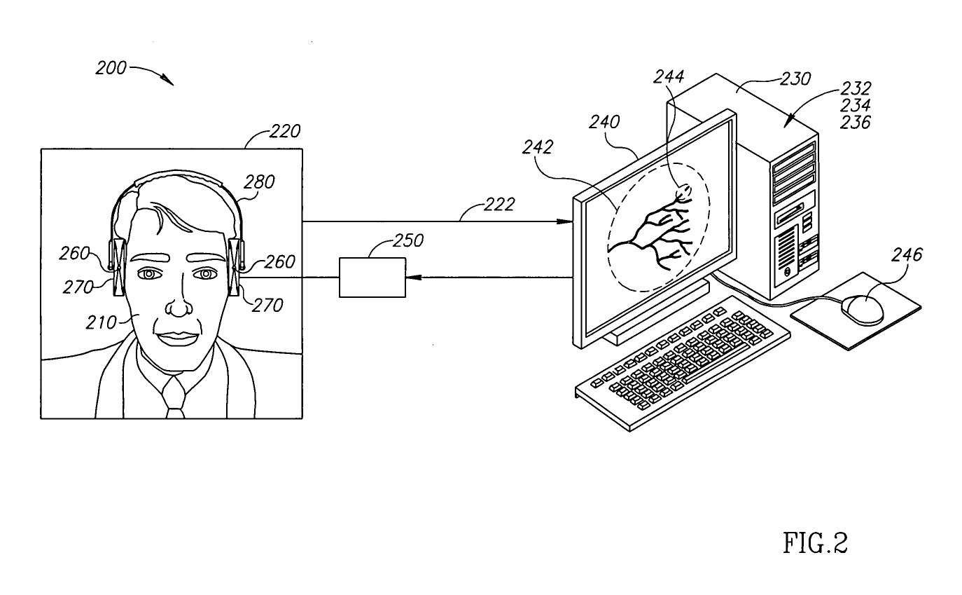

FIG. 2 is a schematic representation of an imaging system 200 according to exemplary embodiments of the invention. Exemplary system 200 includes both one or more volumetric imaging components 220 (e.g. a CTA module) and one or more TCDUS components 270.

One commercially available TCDUS component suitable for use in the context of the invention is the Multigon Neurovision 500 (Yonkers N.Y., USA). One of ordinary skill in the art will be capable of selecting commercially available TCDUS components and incorporating them into the context of the invention. In an exemplary embodiment of the invention, a pencil beam mechanically aimed TCDUS unit is used. Alternatively or additionally, a phase array-steered unit is used. Alternatively or additionally, a focused beam is used.

In some exemplary configurations of system 200, subject 210 is positioned within non-ultrasound volumetric imaging components 220 (e.g. a CT gantry) while wearing TCDUS components 270.

In other exemplary configurations of system 200, subject 210 is subjected to volumetric imaging by imaging components 220 and TCDUS components 270 are subsequently fitted to the cranium.

Non-ultrasound imaging components 220 produce an output image, optionally as a series of planar sections.

Output 222 is relayed to analytic circuitry 230 (pictured as a computer embodiment) which optionally generates a volumetric map 242 of the vasculature.

In an exemplary embodiment of the invention, analytic circuitry 230 includes a segmentation module 232 configured to identify blood vessels in output 222 and to differentiate the blood vessels from adjacent tissue (e.g. bone, brain tissue). Optionally, segmentation module 232 analyzes individual planar sections and/or a volumetric composite of output 222.

In an exemplary embodiment of the invention, analytic circuitry 230 includes a registration module 234 configured to register the volumetric vascular map 242 onto a grid of position co-ordinates, optionally absolute position co-ordinates. In an exemplary embodiment of the invention, TCDUS components 270 are registered onto a same grid or grids. Optionally, a marker that is visible using the imaging component is used, for example, a radio-opaque marker 260.

In an exemplary embodiment of the invention, analytic circuitry 230 includes a target detection module 236 configured to identify targets 130 on the vascular map. One or more targets 244, such as clots and stenoses are characterized by an abnormal reduction in vessel dimension relative to adjacent sections of the same blood vessel. Alternatively or additionally a complete or nearly complete blockage is identified as an abrupt end point in the vasculature.

Optionally, detection module 236 is configured to automatically analyze vessel dimensions and indicate 130 targets 244.

Optionally, detection module 236 is configured to receive user input from an input device 246 (e.g. computer mouse) indicating 130 targets 244. In an exemplary embodiment of the invention, the user selects targets by looking at the vascular map 242. Optionally, a user can view and/or veto and/or add to automatically detected target(s).

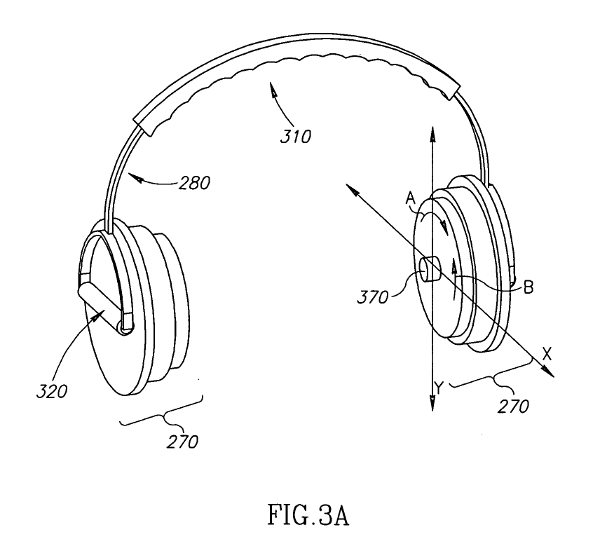

In an exemplary embodiment of the invention, targets 244 are defined in terms of position co-ordinates provided by registration module 234. These position co-ordinates are translated into aiming instruction by controller 250 which aims 140 transducers 370 (FIG. 3B) located in TCDUS units 270.

According to various preferred embodiments of the invention, controller 250 may be electronic, mechanical or electromechanical. Controller 250 may optionally be integrated into analytic circuitry (e.g. computer 230) and linked to one or more motors 390 and/or 392 via an appropriate interface.

In an exemplary embodiment of the invention, controller 250 receives location data from volumetric map 242 including one or more defined targets 244 as an input. Each target 244 is defined as a location in a 3D co-ordinate system of the map produced, for example, by a CT or MRI image acquisition device 220. Optionally, controller 250 keeps track of the effect of therapy of previously identified targets. Alternatively or additionally, controller 250 optionally uses a previously generated map of blockages (e.g., from a check-up of the patient). Optionally, old blockages are ignored in a current treatment.

In an exemplary embodiment of the invention, individual locations of targets 244 are registered with respect to one or more transducers 370 of TCDUS units 270 by registration module 234 and provided to controller 250 as relative locations (with respect to transducers 370).

In an exemplary embodiment of the invention, controller 250 translates these input locations to aiming instructions in the form of directional designations. The directional designations are defined at least in terms of an angle of rotation with respect to the X axis and an angle of rotation with respect to the Y axis. Optionally, the aiming instructions also include displacement information. Displacement information may be provided, for example, as signal amplitude, with a greater signal amplitude corresponding to a greater distance.

The directional designations are translated into mechanical or electronic signals which are relayed to motors 390 and/or 392 which rotate transducer 370 through the appropriate X and Y angles with respect to pivot point 372. Instructions concerning signal amplitude, if supplied, are transmitted electronically from controller 250 to transducer(s) 370.

In an exemplary embodiment of the invention (e.g. FIG. 2), controller 250 is provided as a separate unit and communicates over a distance with motors 390 and/or 392. Communication over distance may be effected, for example by physical connections (e.g. wires or optical fibers) or by cordless communications (e.g. infra-red or radio frequency signals).

In other embodiments of the invention (not pictured), controller 250 is located in TCDUS unit 270 and motors 390 and/or 392 are integrated into controller 250. Optionally, the controller is integrated into a unit worn by a patient, for example a headset.

FIG. 3A illustrates an exemplary headset 280 adapted to position a pair of TCDUS units 270 adjacent to, ears of subject 210 and substantially aligned with a temporal window surface. The temporal window surface is an area on the skull near the ear characterized by relatively narrow bone thickness. Optionally, the narrow bone thickness contributes to more efficient ultrasound penetration. An optional handle 310 may be used to position headset 280 on the head of subject 210. In the pictured embodiment, each TCDUS unit 270 is coupled to headset 280 be means of a pivot axis 320. Each TCDUS unit 270 contains an ultrasonic transducer element 370 containing one or more of transducers. Optionally, the TCDUS units are located and powered and/or positionable so that they can cover at least 30%, at least 50%, at least 70%, at least 90%, or intermediate or greater percentages of the brain volume.

Adjustment of initial position of TCDUS units 270 may be made by manipulating one or more of headset 280, handle 310, axes 320 and TCDUS units 270.

FIG. 3B illustrates an exemplary TCDUS unit 270 in cross section. In the pictured embodiment, transducer 370 is operatively coupled to motors 390 and 392 which are in turn operatively coupled to controller 250 (FIG. 2), optionally via micro-switches 394 and 396.

Optionally, motor 390 controls angular displacement of transducer 370 with respect to the Y axis (e.g. up and down or floor to ceiling orientation) and motor 392 controls angular displacement of transducer 370 with respect to the X axis (e.g. front to back with respect to subject 210). A single angle θ is depicted and includes both θX and θY components with respect to pivot point 372.

Transducer 370 and motors 390 and/or 392 are depicted as being contained within a housing 360 covered by an optionally removable rear cover 362 which faces outwards when TCDUS is placed on a head of a subject. Optionally, motors 390 and/or 392 are step control motors, optionally DC motors.

In an exemplary embodiment of the invention, transducer 370 is mounted in a gimbal. Optionally, motors 390 and 392 are AC gear motors which act in concert to operate a two tangent axis gimbal mechanism. In an exemplary embodiment of the invention, an encoder and 12/24 V controller are provided separately for each of X and Y axes. Optionally, the two axes are connected through precision miniature sealed ball bearings. In an exemplary embodiment of the invention, this arrangement contributes to accurate angular control and/or repeatability of the transducer 370.

In an exemplary embodiment of the invention, motors 390 and/or 392 are configured to rotate the gimbal through 100 to 180 degree with respect to each of the X and Y axes.

In an exemplary embodiment of the invention, gimbals in a pair of TCDUS units 270, are each independently and concurrently controlled by controller 250.

In the depicted embodiment, a transducer cable 374 and a motor cable 376 enter housing 360 through cover 362. Reference 376 is used both for the cable entering the housing and for the split cable, with some of the intervening cable hidden. Optionally, one or more of the cables is provided with a flexible connector 378. Cables 374 and 376 function to supply power and also function as a communication interface with controller 250, if needed (e.g., optionally the motors include a position encoder which reports to the controller). In some cases communication and power supply are handled by separate wires, although single cables are pictured for clarity. A flat connector and/or a bus design may be used.

In order to permit efficient transmission of ultrasonic energy from transducer 370 into the body of a subject, transducer 370 can be immersed in a coupling media 384. The coupling media 384 may be, for example, a gel, an oil (e.g. a silicon based oil) or an aqueous solution. However, the coupling media can interfere with function of and/or cause damage to, motors 390 and 392 and/or micro switches 394 and 396.

Optionally, in order to isolate motors 390 and 392 and/or micro switches 394 and 396 from coupling media 384, a flexible isolation membrane 382 is optionally deployed on a proximal surface of housing 360. In the depicted exemplary embodiment of the invention, isolation membrane 382 is provided as a part of a disposable cushion including coupling media 384 and diaphragm 380. Membrane 382 and diaphragm 380 can be attached to one another by an attachment ring 364. In an exemplary embodiment of the invention, attachment ring 364 is adapted to connect the disposable cushion to a proximal side of housing 360. Optionally, provision of coupling media 384 as disposable cushions permits rapid transfer of TCDUS 270 from one patient to another and/or reduces a need for cleaning TCDUS 270 between patients.

In an exemplary embodiment of the invention, transducer 370 is inserted into coupling media 384 through a breakable or expandable seal 368 in membrane 382. Seal 369 may include, for example, an expandable latex ring. In this way, transducer 370 can be immersed in coupling media 384 while membrane 382 isolates mechanical and/or electronic components of TCDUS 270 from coupling media 384.

Optionally, vacuum hose(s) 350 are provided to apply a vacuum between a proximal side of diaphragm 380 and a head of subject 210. In an exemplary embodiment of the invention, vacuum reduces the need for coupling media between diaphragm 380 and subject 210. Optionally, only a small amount, optionally no coupling media is placed between diaphragm 390 and subject 210. Optionally, hose 350 is fitted with a seal that passes air to the vacuum source, but not fluids or gel.

In an exemplary embodiment of the invention, the aim of the TCDUS components 270 is calibrated by identifying a location on the image (e.g., CT image) expected to have a detectable blood flow, and determining that a blood flow is detected by the TCDUS. Optionally, a plurality of such locations are selected, for example, 3 points along the circle of Willis.

Generating a Volumetric Map

FIG. 2 illustrates a volumetric map 242 of cerebral vasculature displayed upon a display 240. A map of this type may be generated, for example using computerized tomography (CT) or Magnetic Resonance Imaging (MRI). In an exemplary embodiment of the invention, the volumetric map 242 indicates vessel width (e.g., internal diameter). Optionally, vessel width is indicated by contrast material employed in CTA or MRA. Volumetric maps of this general type operate segmentation algorithms on input data in order to identify tissue of various types. Optionally, scanning with TCDUS units 270 provides blood flow data which is registered on map 242.

Examples of segmentation algorithms suitable for use in the context of the present invention may be found, for example, in an article entitled “Medical Image Computing and Computer-Assisted Intervention” in Lecture notes in Computer Science (2003); published by Springer Berlin/Heidelberg (2879/2003: 562-569; ISSN: 0302-9743), the disclosure of which is fully incorporated herein by reference. However, other methods may be used as well.

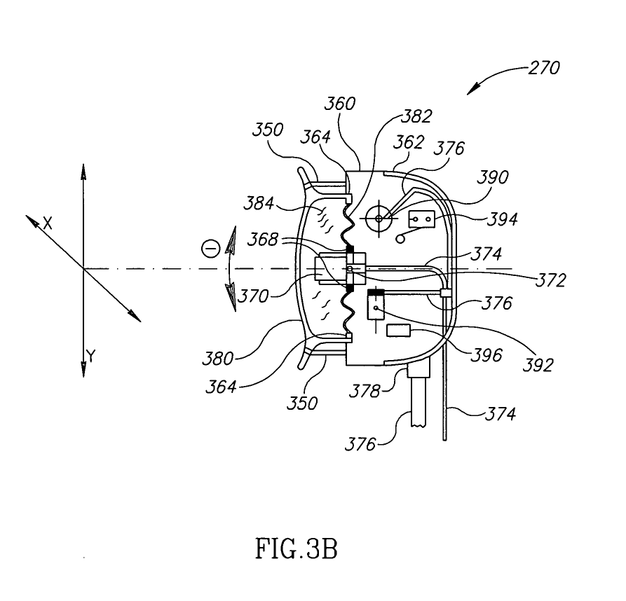

FIG. 4 is a transverse longitudinal section 400 of a brain 460 with a vascular map and ultrasound unit according to an exemplary embodiment of the invention superimposed thereon. A reference 450 indicates a nose, for orientation purposes.

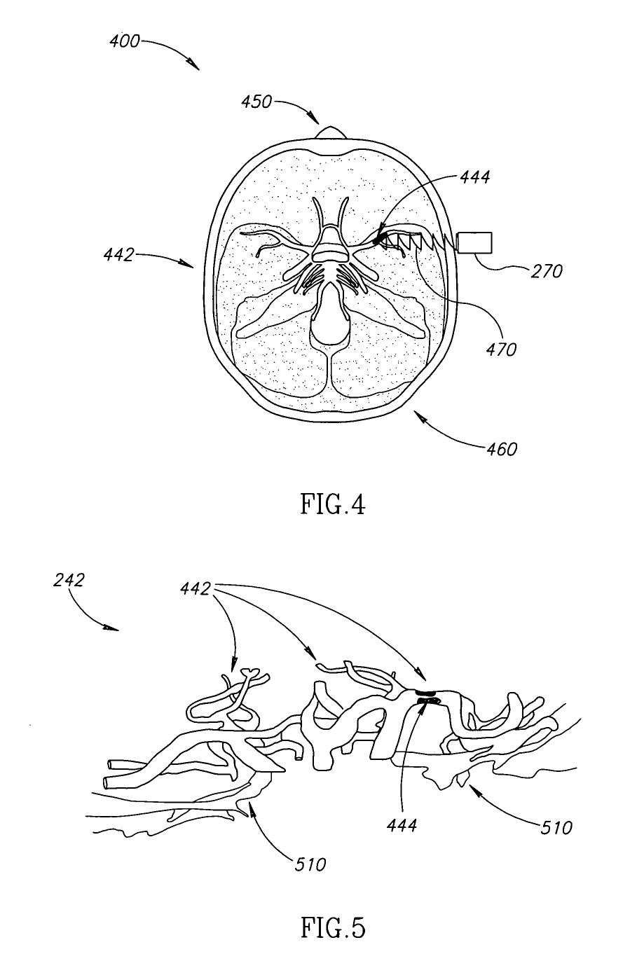

FIG. 5 schematically illustrates map 242 in greater detail. Blood vessels 442 are differentiated from bones 510. A selected target 444 in a blood vessel is indicated. Selection of target 444 may be manual or via analytic circuitry as described above. In an exemplary embodiment of the invention, CT is employed to generate map 242; optionally CT angiography provides a map 242 which indicates a blood volume in blood vessels 442 by gauging an amount of contrast material. Optionally, MRA is substituted for CT angiography as a means of providing blood volume data.

Identifying Targets

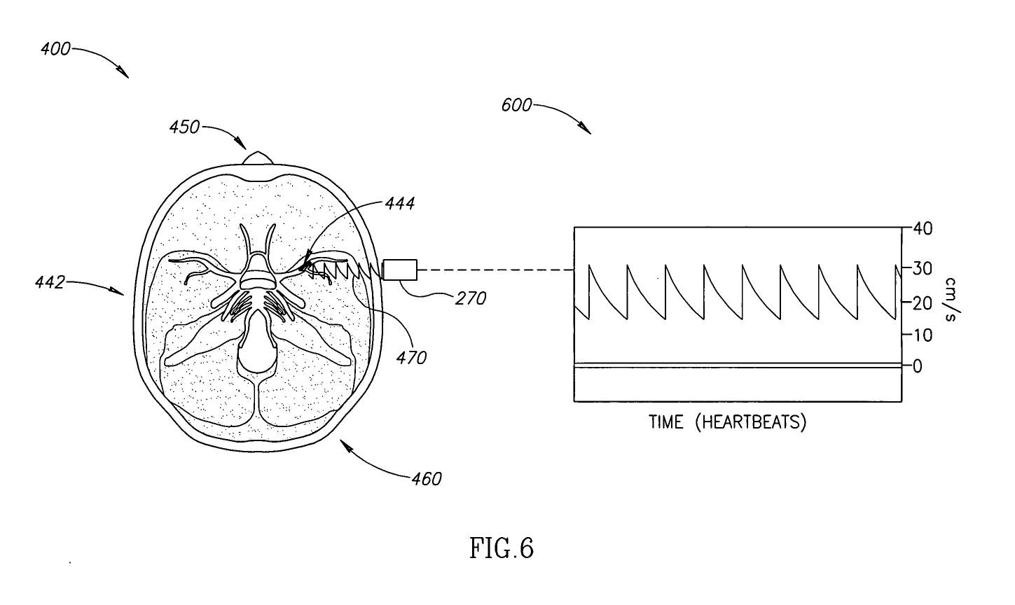

FIGS. 4, 5 and 6 illustrate identification of targets 444. Although a single target 444 is depicted for clarity, multiple targets 444 may optionally be identified concurrently. FIG. 6 repeats the crosses sectional view of FIG. 4 and adds a chart 600 indicating an exemplary measured signal. In FIGS. 4 and 6, ultrasound energy is depicted as a wave 470 impinging on target 444. Such targets may be treated, for example, in parallel (e.g., separate TCDUS transmitters), in series or using time-sharing (e.g., a round-robin method).

In an exemplary embodiment of the invention, automated identification 130 of targets is conducted by target detection module 236. Optionally, automated target identification 130 relies upon blood volume data as described above.

Optional Addition of Flow Data to the Map

In some exemplary embodiments of the invention targets may be confirmed using blood flow data provided by TCDUS scanning.

In those embodiments of the invention which employ TCDUS scanning, the scanning can be performed using a multigating procedure such as “step and shoot” or “on the fly”. Optionally, the scan is a volume scan in which all depth ranges relative to the transducer (e.g., within a given range) are sampled.

In “step and shoot” scanning, transducer 370 is held at a fixed angle and data is acquired from multigates at half second intervals until all depths have been evaluated. At that point, the transducer is stepped to the next angle. The process can be repeated until a desired portion of the brain volume is imaged.

In “on the fly” scanning, the transducer is rotated at a constant angular velocity (e.g. 2 degrees/sec) and Doppler flow data is acquired on the fly from multiple time gates.

Both “step and shoot” scanning and “on the fly” scanning produce 3D Doppler max flow maps (optionally shown color coded).

Manual Target Identification

In other embodiments of the invention, an operator of system 200 identifies targets 444 manually on a display screen 240 using an input device (e.g. mouse 246) to indicate each target 244 on map 242. Optionally, manual identification relies upon anatomic data, for example anatomic data from CTA or MRA.

In an exemplary embodiment of the invention, a preliminary manual identification of a suspected target 244 or 444 is made. Optionally, TCDUS 270 is aimed so that transducer 370 progresses linearly along blood vessel 442 from a short distance before the suspected target (e.g. 2-3 mm) to a short distance after the suspected target. This provides a plot of flow velocity as a function of linear displacement. Suspected target 444 is confirmed as being at the point where there is a distinct change in flow rate.

### Determining Target Co-ordinates

In an exemplary embodiment of the invention, registration between volumetric map 242 and TCDUS flow data is achieved by placing markers (e.g., 260 in FIG. 2) of various types known to those in the art of image registration on TCDUS unit 270 at a known displacement from transducer 370. Suitable marker types include, but are not limited to, magnetic, optical and fiducial markers. Optionally, the markers are visualized in the CT image, for example being radio-opaque. The markers are used to register CT and TCDUS data to a common 3D co-ordinate system. Optionally, the markers are active or reflective for ultrasound.

Aiming Transcranial Doppler Ultrasound

FIGS. 3A and 3B illustrate exemplary means for aiming transducer 370 of TCDUS 270.

In an exemplary embodiment of the invention, transducer 370 is mounted in a gimbal with a pivot point 372. The gimbal is capable of angular displacement of transducer 370 relative to pivot point 372 in at least an X and a Y direction as indicated by arrows in FIGS. 3A and 3B. Angular displacement may optionally be provided by motors 390 and/or 392 or by actuators controlled by an external motor.

Transducer Types

In an exemplary embodiment of the invention, TCDUS unit 270 employs pulsed wave transducers 370 which can provide information from a plurality of distances/depths.

Pulsed wave transducers typically employ the same crystal to alternately send and receive an ultrasonic signal. Pulsed wave probes are characterized by a specific sample volume (width) and a depth. The depth can be varied by varying Pulse repetition frequency (PRF) and/or time.

Pulsed wave probes are useful for differentiation of blood vessels and/or in cases where overlying blood vessels and a transmitted ultrasonic wave lie on the same line as the blood vessel being measured.

In an exemplary embodiment of the invention, transducer 370 has a diameter of 12, optionally 15, optionally 17, optionally 20, optionally 22 mm or lesser or greater or intermediate diameters.

Transducer Considerations

In an exemplary embodiment of the invention, an ultrasonic signal with a frequency in the range of 1 MHz to 16 MHz is employed. In general, lower frequency probes are used to insonate deeper vessels or penetrate the bone and higher frequency probes are used for more superficial vessels.

For example, a 2 MHz probe is often employed for intracranial circulation (ICA); a 4 MHz probe is often employed for extracranial circulation and/or analysis of large peripheral vessels; a 8 MHz probe is often employed for extracranial circulation, ophthalmic artery analysis, or small peripheral vessel examination and a 16 MHz probe is often employed for intraoperative examination of exposed vessels in the brain and/or heart.

During intra-operative use, a probe may be placed directly on the exposed blood vessel.

In an exemplary embodiment of the invention, transducer 370 is characterized by a power output of less than 500, 400, 300, 200, 100 mw/cm² or lesser or greater values.

In an exemplary embodiment of the invention, transducer 370 is characterized by a range of up to 1 cm, 5 cm, 7 cm, 9 cm, 12 cm, 15 cm or smaller or intermediate values.

In an exemplary embodiment of the invention, transducer 370 may be subjected to an angular displacement of ±90, ±60, ±45, ±30, ±15 degrees or intermediate values in the X and/or Y direction.

In some cases, an angle of incidence between ultrasonic energy emanating from transducer 370 and blood flow in a vessel, is unknown. In an exemplary embodiment of the invention, the determined flow velocity depends on a cosine of this angle of incidence. Optionally, two, or three transducers 370 aimed at a same target 444 contribute to a more accurate calculation of flow velocity and/or direction. Optionally, comparison of data for a single target 444 from two or more transducer 370 permits determination of the angle of incidence for each transducer. Optionally, imaging data is used to estimate the angle and correct the TCDUS velocity estimation.

In cranial applications, a pair of TCDUS units mounted substantially aligned with ears of subject 210 are generally sufficient to permit Doppler shift data for any target 244 within the cranium.

In some embodiments of the invention, the TCDUS is also used to obtain a vascular map, for example, by scanning all of the brain. Optionally, a non-Doppler modality is used. Optionally, the ultrasonic scanning replaces the non-ultrasonic imaging method or is used to detect that the image has changed substantially and a new image needs to be acquired.

Safety

TCDUS is a non-invasive method which enables physicians to define moment-to-moment changes in cerebral blood flow velocities.

The rapid response time of TCDUS permits a skilled operator and/or automated system to react to changes as they occur. Once transducer 370 of TCDUS unit 270 is aimed at a target 444, the flow rate at the target is output substantially in real time. In sharp contrast CT or MRI scan the cranium for a period of minutes and present an image of a specific target only after the scan is complete.

In some cases, TCDUS is more sensitive than image based analytic methods because TCDUS analyzes blood flow as opposed to vessel diameter. Optionally, a change in vessel diameter which is not visibly perceptible causes a change in blood velocity which can be measured by ultrasound. In an exemplary embodiment of the invention, TCDUS permits detection of as little as 30%, 20%, 10% or 5% increase in blood flow at the target.

Optionally, TCDUS monitoring of recanalization can reduce the amount of tPA needed for full recanalization by accelerating tPA activity and/or by providing substantially real time monitoring. In an exemplary embodiment of the invention, substantially real time monitoring reduces the chance that tPA will continue to be administered after recanalization is complete.

In some cases, TCDUS, due to its small size, permits intra-operative and post-operative monitoring to identify hypoperfusion, preoperative thrombosis, hyperperfusion syndrome and ongoing intra-arterial embolism. These possibilities significantly reduce patient risk before, during and after surgery. In an exemplary embodiment of the invention, a TCDUS beam is re-aimed automatically, if during surgery the brain shifts. Optionally, if a current target disappears (e.g., Doppler readings change significantly from expected), the beam scans a volume around the original aiming volume, to detect the shifted location of the blood vessel. It is noted that the TCDUS beam generally does not interfere with surgical tools and/or harm physicians, even if aimed directly at them.

TCDUS is generally considered safer than other medical imaging techniques because it typically does not employ contrast material. This significantly reduces the risk of allergic reactions and kidney problems and decreases risk to the patient.

Alexandrov A V and Joseph M (Transcranial Doppler: an overview of its Clinical Applications (2000) Internet Journal of Emergency and Intensive Care Medicine 4(1):http://www/ispub.com/journals/IJEICM/vol4N1tcd.htm—accessed Aug. 1, 2006) reported that a technology assessment report published in 1990 by The American Academy of Neurology indicates that TCD has established value in the assessment of patients with intracranial Stenosys, Collaterals, Subarachnoid Hemorrhage, and Brain Death. Alexandrov and Joseph's overview of clinical applications of TCD has been approved by the Board of Directors of the American Society of Neuroimaging and the Neurosonology Research Group of the World Federation of Neurology. The disclosure of these documents is incorporated herein by reference.

IQ Spectrum Analysis

In an exemplary embodiment of the invention, a spectrum analysis is performed on the Doppler shift signal. Optionally, the time domain samples inphase (I) and quadrature (Q) complex signals representing the Doppler data The result is optionally shown (e.g., per location on the vascular image) as a color coded, blood flow spectrum display where the X axis indicates time, the Y axis indicates Doppler shift (kHz) which corresponds directly to velocity (cm/sec) and the Z axis (color) indicates intensity of energy reflection. Each point under the curve represents relative amount of energy of red blood cells flowing at a certain velocity at a certain point in time. In an exemplary embodiment of the invention, selecting a location on the vascular image commands the TCDUS to aim at the point. Optionally, non-Doppler imaging methods are employed by the ultrasonic system.

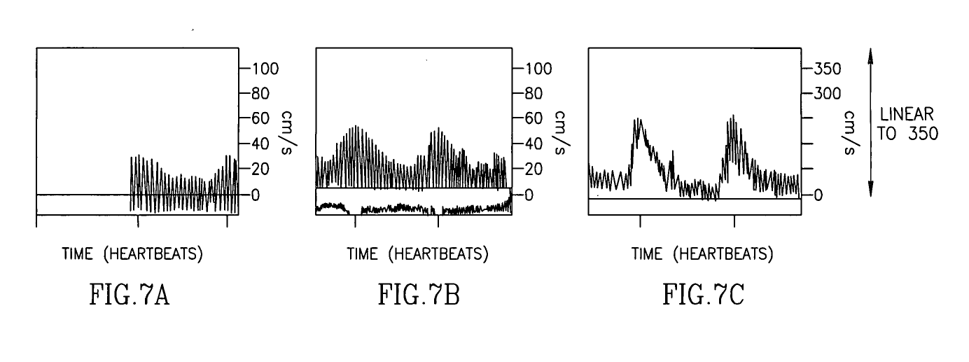

Exemplary outputs of flow maxima of spectrum analysis from TCDUS are depicted in FIGS. 6, 7A, 7B and 7C.

FIG. 6 depicts a diagnostic spectrum analysis by TCDUS in a patient with a partially occluded vessel 442. Peak blood flow is 30 cm/s at the occlusion.

FIG. 7A depicts a diagnostic spectrum analysis by TCDUS in a patient with a partially occluded vessel 442 characterized by a peak blood flow of 30 cm/s. This figure was taken after 12 minutes of monitoring.

FIG. 7B depicts a diagnostic spectrum analysis by TCDUS in the same patient 30 seconds after FIG. 7A. Peak blood flow has increased to 60 cm/s.

FIG. 7C depicts a diagnostic spectrum analysis by TCDUS in the same patient 6 hours later monitored according to an exemplary embodiment of the invention. Peak blood flow has increased to 300 cm/s.

In an exemplary embodiment of the invention, results of spectral analysis from TCDUS are presented to a user of as flow maxima as in FIGS. 6, 7A, 7B and 7C. In other embodiments of the invention, results are presented to a user with the Z axis color information.

In an exemplary embodiment of the invention, controller 250 directs transducer 370 of TCDUS unit 270 to scan along a blood vessel identified in map 242. Optionally, a maximum flow or average flow is displayed for each displacement co-ordinate along the blood vessel. Because each point in the blood vessel is defined as a set of 3D co-ordinates, this method can be termed “3D scan conversion”. Optionally, 3D scan conversion is employed to locate and/or confirm targets 244. Optionally, a 3D reconstruction of maximum flow data from all directions is performed to generate a 3D map image which can be registered with respect to an anatomic map produced by non-ultrasound means.

### Optional Use in Conjunction With Therapy

Referring again to FIGS. 7A, 7B and 7C, a system 200 according to exemplary embodiments of the invention may be employed in conjunction with a therapeutic regimen. For example, a thrombolytic drug (e.g. tissue plasminogen activator; tPA) may be delivered systemically to dissolve clots which form targets 244. It is known that ultrasonic energy applied to clots works synergistically with thrombolytic drugs.

In an exemplary embodiment of the invention, low energy TCDUS is applied to target 244 concurrently with systemic delivery of a thrombolytic drug.

FIG. 7A shows blood flow as a function of time at target 244 prior to onset of therapy effect.

FIG. 7B shows blood flow as a function of time at target 244 thirty seconds after onset of therapy effect.

FIG. 7C shows blood flow as a function of time at target 244 six hours later.

This series of figures illustrates the ability of a system 200 according to exemplary embodiments of the invention to aid in local monitoring at targets 244 of therapy with thrombolytic drugs delivered systemically. In an exemplary embodiment of the invention, ultrasonic energy delivered by the TCDUS to targets 244 acts additively, optionally synergistically, with the thrombolytic drugs.

Exemplary embodiments of the invention rely upon execution of various commands and analysis and translation of various data inputs. Any of these commands, analyses or translations may be accomplished by software, hardware or firmware according to various embodiments of the invention. In an exemplary embodiment of the invention, machine readable media contain instructions for translation of 3D position co-ordinates into aiming instructions for a transducer 370 of a TCDUS unit 270 and/or registration of a location of transducer 370 of a TCDUS unit 270 onto a volumetric map of cerebral vasculature 242 are provided.

In an exemplary embodiment of the invention, circuitry (e.g. circuitry of computer 230) executes instructions for translation of 3D position co-ordinates into aiming instructions for a transducer 370 of a TCDUS unit 270 are provided and/or instructions for registration of a location of transducer 370 of a TCDUS unit 270 onto a volumetric map of cerebral vasculature 242.

The present invention has been described using detailed descriptions of embodiments thereof that are provided by way of example and are not intended to limit the scope of the invention. In particular, numerical values may be higher or lower than ranges of numbers set forth above and still be within the scope of the invention. The described embodiments comprise different features, not all of which are required in all embodiments of the invention. Some embodiments of the invention utilize only some of the features or possible combinations of the features. Alternatively or additionally, portions of the invention described/depicted as a single unit may reside in two or more separate physical entities which act in concert to perform the described/depicted function. Alternatively or additionally, portions of the invention described/depicted as two or more separate physical entities may be integrated into a single physical entity to perform the described/depicted function. Variations of embodiments of the present invention that are described and embodiments of the present invention comprising different combinations of features noted in the described embodiments can be combined in all possible combinations including, but not limited to use of features described in the context of one embodiment in the context of any other embodiment. Specifically, features described in the context of a method may be present in an apparatus or system and features described in the context of an apparatus or system may be present in a method. The scope of the invention is limited only by the following claims.

In the description and claims of the present application, each of the verbs “comprise”, “include” and “have” as well as any conjugates thereof, are used to indicate that the object or objects of the verb are not necessarily a complete listing of members, components, elements or parts of the subject or subjects of the verb.

All publications and/or patents and/or product descriptions cited in this document are fully incorporated herein by reference to the same extent as if each had been individually incorporated herein by reference.

1. A medical diagnostic system, the system comprising:
a) a non-ultrasound imaging module configured to generate a volumetric vascular map;
b) a controller configured to translate a 3D position co-ordinate from the map into an aiming instruction; and
c) a trans-cranial motion-detecting ultrasound unit adapted to aim a narrow beam of a transducer responsive to the aiming instruction.

2. A system according to claim 1, comprising:
e) a target identification module.

3. A system according to claim 2, wherein the target identification module operates automatically to identify targets in the volumetric map.

4. A system according to claim 2, wherein the target identification module responds to a user indication of targets in the volumetric map.

5. A system according to claim 2, wherein the controller formulates the aiming instruction responsive to a target identified by the target identification module.

6. A system according to claim 1, wherein the imaging module employs computerized tomography (CT).

7. A system according to claim 1, wherein the imaging module employs magnetic resonance induction (MRI).

8. A system according to claim 1, wherein the imaging module employs CT angiography (CTA).

9. A system according to claim 1, wherein the imaging module employs MRI angiography (MRA).

10. A system according to claim 1, wherein the vasculature includes cerebral vasculature.

11. A system according to claim 1, wherein the motion-detecting ultrasound unit is a transcranial Doppler ultrasound unit (TCDUS).

12. A system according to claim 1, wherein the controller is adapted to scan said beam in space according to said map.

13. A system according to claim 1, comprising a medication providing system whose activity is coordinated with said motion-detecting ultrasound unit.

14. A medical diagnostic method, the method comprising:
a) generating a volumetric map of a portion of a vasculature system using a non-ultrasound imaging modality;
b) registering a position of a motion-detecting ultrasound transducer with respect to the volumetric map;
c) identifying a target on the map by a 3D position co-ordinate;
d) translating the 3D position co-ordinate into an aiming instruction;
e) aiming a pencil beam of a trans-cranial motion-detecting ultrasound transducer at the target responsive to the aiming instruction; and
f) acquiring hemodynamic data pertaining to the target.

15. A method according to claim 14, wherein the identifying is performed by analytic circuitry.

16. A method according to claim 14, wherein the identifying is performed by a user.

17. A method according to claim 14, wherein the volumetric map is generated from computerized tomography (CT) data.

18. A method according to claim 14, wherein the volumetric map is generated from magnetic resonance induction (MRI) data.

19. A method according to claim 14, wherein the volumetric map includes blood flow data.

20. A method according to claim 19, wherein the blood flow data is provided by CT angiography (CTA).

21. A method according to claim 19, wherein the blood flow data is provided by MRI angiography (MRA).

22. A method according to claim 14, wherein the volumetric map depicts cerebral vasculature.

23. A method according to claim 14, wherein the motion-detecting ultrasound transducer is a transcranial Doppler ultrasound unit (TCDUS).

24. A therapeutic method, the method comprising:
a) generating a volumetric map of a portion of a vasculature system of a subject using a non-ultrasound imaging modality, the map including preliminary blood flow data;
b) registering a position of a motion-detecting ultrasound transducer with respect to the volumetric map;
c) identifying a target on the map by a 3D position co-ordinate responsive to the preliminary blood flow data;
d) translating the 3D position co-ordinate into an aiming instruction;
e) aiming a pencil beam of a trans-cranial motion-detecting ultrasound transducer responsive to the aiming instruction; and
f) acquiring hemodynamic data pertaining to the target concurrent with systemic administration of a thrombolytic drug.

25. A system according to claim 1, wherein said trans-cranial motion-detecting transducer is mounted in an apparatus for reducing signal interference in an ultrasound examination, the apparatus comprising;
a) a cushion adapted to conform to the skull temporal window surface and to an ultrasonic transducer; and
b) a quantity of a first ultrasound coupling media contained within the cushion at a pressure selected to allow the conforming;
wherein the conforming to the skull temporal window surface and the conforming to the ultrasound transducer preclude the formation of air pockets capable of significantly interfering with ultrasonic transmission.

26. An apparatus according to claim 25, wherein the first ultrasound coupling media includes an oil.

27. An apparatus according to claim 25, wherein the first ultrasound coupling media includes a gel.

28. An apparatus according to claim 25, wherein the first ultrasound coupling media includes water.

29. An apparatus according to claim 25, comprising:
a second ultrasound coupling media applied to the first surface.

30. An apparatus according to claim 25, comprising:
a second ultrasound coupling media applied to the second surface.

31. An apparatus according to claim 29, wherein the second ultrasound coupling media includes a gel.

32. An apparatus according to claim 25, comprising an ultrasound transducer ultrasonically coupled to said cushion and a motor adapted to physically move said ultrasonic transducer.

33. A medical diagnostic system, the system comprising:
a) a transcranial motion-detecting ultrasound unit including at least one transducer, the unit attached to a cranium and adapted to scan a brain volume and generate a 3D volumetric vascular map of at least part of the cranium;
b) a controller configured to translate a 3D position co-ordinate from the map into an aiming instruction; and
c) an aiming mechanism adapted to aim a transcranial motion-detecting transducer responsive to the aiming instruction.

34. A system according to claim 1, comprising a controller configured to maintain said aiming for several hours.

35. A system according to claim 1, comprising:
an input for providing a plurality of targets in said map; and
a controller configured to sequentially monitor a plurality of different targets using said aimed pencil beam.

36. A system according to claim 1, comprising a registration module configured to register a position of the transducer with respect to the volumetric map.
